(19) United States
(12) Patent Application Publication
(10) Pub. No.: US 2009/0149958 A1
(43) Pub. Date: Jun. 11, 2009

(54) STRUCTURALLY REINFORCED SPINAL NUCLEUS IMPLANTS

(76) Inventors: Ann Prewett, Bloomfield Hills, MI (US); Gerald Gontarz, Spotswood, NJ (US)

Correspondence Address: CARTER, DELUCA, FARRELL & SCHMIDT, LLP, 445 BROAD HOLLOW ROAD, SUITE 420, MELVILLE, NY, 11747, US

(21) Appl. No.: 12/263,797

(22) Filed: Nov. 3, 2008

Related U.S. Application Data
(60) Provisional application No. 61/001,432, filed on Nov. 1, 2007.

(51) Int. Cl. A61F 2/44; B32B 38/08

(52) U.S. Cl. 623/17.16; 156/145

(57) ABSTRACT

A spinal nucleus implant is provided which includes a braided three-dimensional reinforcement member having a polymeric matrix imbued therein, the implant configured to have a shape consistent with a cavity within an intervertebral disc space. The polymeric matrix may be a fluid absorbing polymer, e.g., a hydrogel or a substantially non-fluid absorbing in-situ curable elastic polymer. A method of making a spinal nucleus implant is provided which includes providing a braided three-dimensional reinforcement member configured and dimensioned to have a shape consistent with a cavity in an intervertebral space and infusing the braided three-dimensional reinforcement member with a liquid polymer capable of forming a polymeric matrix. Also provided is a spinal nucleus implant including a three-dimensional reinforcement member adapted and configured to undergo anisotropic expansion, the implant configured to have a shape consistent with a cavity within an intervertebral disc space. A method of treating a degenerating intervertebral disc includes creating an incision in an annulus; removing at least a portion of a nucleus pulposus; and inserting, through the incision, a spinal nucleus implant according to the present disclosure.

### CROSS-REFERENCE TO RELATED APPLICATIONS

This application claims priority of U.S. Provisional Application Ser. No. 61/001,432 filed Nov. 1, 2007 and is incorporated herein by reference in its entirety.

### BACKGROUND

1. Technical Field

Repair of spinal defects and/or degenerative diseases of the spine.

2. Background of Related Art

The spinal disc, in concert with surrounding muscle, ligament and other structures, supports the loads of the lumbar spine generated by the body. The spinal disc is comprised of a fibrous tissue-like annular ring which in turn is composed largely of Type I collagen and an internal material of high water content, known as the nucleus pulposus. As a result of the aging process, the nucleus pulposus may degenerate and lose its high water content. As the fluid-like component of the disc diminishes, the nucleus becomes increasingly fibrous in nature. The annulus which surrounds the nucleus also changes with time and can delaminate or perforate. This process in which age related changes to both the nucleus and annulus occur is often associated with pain and is recognized to be a condition known as degenerative disc disease (DDD). Techniques to reduce or eliminate the pain associated with DDD include removal or partial removal of the nucleus (discectomy), replacement of the nucleus and part of the annulus with a metallic components, and removal of the nucleus and replacement with a biomaterial or natural nuclear substitute (nucleus replacement). If one attempts to replace the native nucleus of a degenerated disc with a substance comprised of high water content but low solids, the material may resemble the native healthy nucleus pulposus but may be prone to herniation through a weak annulus. If the annulus has perforations or tears, it is often unable to prevent passage of nuclear fluid across its surface. Thus, a fluid-like nucleus replacement implant would be prone to leakage and could be pressed through these passages rendering it unsuccessful as an implant. Indeed, high water content, low durometer hydrogels composed of PVA have exhibited high extrusion rates when implanted in the body in the disc space.

Accordingly, a potential shortcoming of artificial disc replacements is the propensity for extrusion of the implant through the annulus. The nucleus pulposus is held in place by the annulus in vivo. However, the annulus must be compromised in order to gain access to the diseased or damaged disc space. The resulting annular defect provides a path of least resistance through which a nucleus replacement or augmenter may travel under extremes of load and/or motion. In the case of implants which are made from a soft material, e.g., a hydrogel from polyvinyl alcohol, the propensity for extrusion through creep or flow is higher as the material gets softer. The likelihood of extrusion also increases with increased load.

Certain structures have been suggested to help prevent extrusion of an artificial nucleus pulposus through an annulus. One approach involves reinforcing the annulus directly such as by placement of a band or patch of material along the inside or outside diameter of the annulus. See, e.g., U.S. Pat. Nos. 7,220,282, 6,805,695, 6,712,853, and 6,592,625. Another approach involves providing an artificial spinal nucleus implant with a retaining or reinforcing structure. See, e.g., U.S. Pat. No. 6,533,817 (a prosthetic disc nucleus having a hydrogel core which is surrounded by a jacket for constraining expansion of the hydrogel core). U.S. Pat. Nos. 5,562,736 and 5,674,295 disclose an implant having a constraining jacket surrounding a hydrogel core. As described therein, a hydrogel material is dehydrated, resulting in an undersized substantially cylindrical gel capsule which is then inserted into the constraining jacket which is then closed to prevent the hydrogel from escaping the confines of the jacket. The implant is rehydrated and conditioned by a series of compressive loads which renders the nucleus body to a partially flattened or oval shape. The implant is then inserted into a retaining tube to maintain the oval shape up until implantation. Alternative embodiments include an outer skin formed by ion implantation which causes outer layer polymerization and functions as the constraining jacket. U.S. Pat. No. 6,022,376 describes an implant made from an amorphous hydrogel polymer core surrounded by a constraining jacket. In one embodiment, the amorphous polymer is poured into one end of the constraining jacket in an unhydrated state, and the jacket then closed. The implant is then massaged to flatten and narrow the implant in preparation for implantation. Alternatively, the amorphous polymer may be injected into the constraining jacket. In one embodiment, an empty constraining jacket is implanted into the disc space and the amorphous polymer is then injected into the constraining jacket. In one embodiment, the amorphous polymer is shaped into a plurality of “microchips” which have been manufactured to have a certain shape. U.S. Pat. No. 6,132,465 is directed to a nucleus implant having a hydrogel core in a constraining jacket. The hydrogel core is inserted into the constraining jacket in a wedge-shaped dehydrated state and then implanted into the nucleus cavity. A final dehydration step is described where the hydrogel core can be forced into certain shapes, i.e., it can be “entirely flat”. U.S. Pat. No. 6,602,291 describes a prosthetic spinal disc nucleus which is made with a hydrogel core having a first shape in the hydrated state. It is then placed in a constraining jacket and reshaped to have a second shape in the dehydrated state. The core is configured to transition from the second shape to the first shape on hydration. U.S. Pat. Nos. 7,204,897 and 6,726,721 describe, inter alia, a spinal disc implant for replacement of at least a part of the nucleus of a intravertebral disc which is composed of at least two essentially parallel soft layers of an elastically deformable hydrogel and at least one rigid layer which is reinforced by a reinforcement component selected from the group consisting of textile fibers, a perforated polymer foil or a combination of the two. In one embodiment, textile fibers are the reinforcing component and have a configuration selected from the group consisting of a knit net, a non-woven textile, or a woven lattice placed parallel to the firm layer surface.

Fiber architectures have been used as structural backbones in certain contexts. Fiber architecture may be knit, woven or braided. A knit structure is made by intertwining yam, monofilaments Or thread in a series of connected loops. Although a two-dimensional knit structure may be amenable to some dimensional malleability, i.e., folding, stretching, compression, etc., a three-dimensional knit structure maintains a relatively fixed shape due to the interconnection of loops on at least three to four sides. A woven structure is made by interlacing or interweaving strips, monofilaments or strands of material. The strips, monofilaments or stands of woven structures also provide a relatively fixed structure, i.e., the angles of interwoven threads are fixed. Braided structures are made by interweaving three or more strands, strips, or lengths of monofilaments in a diagonally overlapping pattern. The various features of fiber architectures are amenable to specific applications. For example, U.S. Pat. No. 4,917,699 is directed to a prosthetic ligament constructed from a three-dimensional braid. U.S. Pat. No. 5,711,960 is directed to a biocompatible implant material comprising a tri-axial or more three-dimensional fabric. As described therein, the implant material comprises a three-dimensional fiber architecture as its basic constituting structure. See column 4. The three-dimensional woven and knitted fabric architecture may be used as is or after coating it with the same or different material. Id. Surface treatment methods are described, e.g., at column 12, et. seq. A fiber fabric body filled and coated with a gel of a cartilage-like hardness is described in Example 2, at column 19. U.S. Pat. No. 7,066,960 is directed to an intervertebral disk replacement. As described therein, an intervertebral disc prosthesis has a matrix of bioincorporable fabric, and a nuclear core centrally mixed into the fabric. The matrix may be fabric made from continuous woven fibers. See column 8.

There is a continuing need for improved spinal implants that can withstand the dynamic rigors of the intervertebral space, especially in circumstances where structural elements of the spine such as the annulus are diseased and/or compromised.

### SUMMARY

A spinal nucleus implant is provided which includes a braided three-dimensional reinforcement member having a polymeric matrix imbued therein, the implant configured to have a shape consistent with a cavity within an intervertebral disc space. The polymeric matrix may be a fluid absorbing polymer, e.g., a hydrogel. The polymeric matrix may also be a substantially non-fluid absorbing elastic polymer. The three-dimensional reinforcement member may be made of a fiber selected from the group consisting of monofilament, multifilament and combinations thereof. In one embodiment, the implant is configured to transform from a first configuration to a second configuration, the first configuration having a smaller cross-section than the second configuration.

Also provided is a method of making a spinal nucleus implant which includes providing a braided three-dimensional reinforcement member configured and dimensioned to have a shape consistent with a cavity in an intervertebral space and infusing the braided three-dimensional reinforcement member with a fluid absorbing polymer. The fluid absorbing polymer can be a hydrogel. In one embodiment, the braided three-dimensional reinforcement member is placed within a mold cavity which has dimensions greater than the braided three-dimensional reinforcement member to allow the fluid absorbing polymer to be absorbed into and saturate the braided three-dimensional reinforcement member and to encapsulate the braided three-dimensional reinforcement member with a layer of fluid absorbing polymer. In another embodiment, the spinal nucleus implant is dehydrated to reduce the dimensions of the implant.

Also provided is a method of treating a degenerating intervertebral disc which includes creating an incision in an annulus; removing at least a portion of a nucleus pulposus; and inserting, through the incision, a spinal nucleus implant which includes a braided three-dimensional reinforcement member having a polymeric matrix imbued therein, the implant configured to have a shape consistent with a cavity within the intervertebral disc space.

Also provided is a spinal nucleus implant including a three-dimensional reinforcement member adapted and configured to undergo anisotropic expansion, the implant configured to have a shape consistent with a cavity within an intervertebral disc space. The three-dimensional reinforcement member may be a braided fabric. The spinal nucleus implant may also include a fluid absorbing polymer, e.g., a hydrogel.

### BRIEF DESCRIPTION OF THE DRAWINGS

FIG. 1 is a schematic top view depicting a spinal nucleus implant containing a three-dimensional reinforcement member going from a compressed configuration to an expanded configuration.

FIG. 2 is a perspective view of a spinal nucleus implant containing a three-dimensional reinforcement member in a compressed configuration.

FIG. 3 is a perspective view of a spinal nucleus implant containing a three-dimensional reinforcement member in an expanded configuration.

FIG. 4 is a top view of a three-dimensional reinforcement member for use in a spinal nucleus implant.

### DETAILED DESCRIPTION OF PREFERRED EMBODIMENTS

A spinal nucleus implant composite (“SNIC”) according to the present disclosure is uniquely suited for implantation and residence in the disc space of a diseased or damaged intervertebral disc. The implant includes a novel interiorly embedded support device. The support device is a braided three-dimensional reinforcement member which utilizes unique capabilities manifest by three-dimensional braid architecture. Three-dimensional braiding techniques allow construction of fiber architectures with a high degree of structural integrity and fiber volume fractions, a wide range of pore geometries and pore distribution, and the unique ability to maintain and/or to selectively limit the outer dimensional configuration of the implant while providing a convenient modality for dimensional compression into a desirable implantation configuration. The braided three-dimensional reinforcement member is anchored in the body of the implant and provides reinforcement to the body of the implant which increases structural integrity, creep resistance and assists in preventing radial bulging of the implant under load bearing conditions.

The braided three-dimensional reinforcement member forms the structural backbone of the SNIC. A polymeric matrix advantageously surrounds the reinforcement member. The polymeric matrix can be made of a polymer which can be biostable, bioabsorbable, biodegradable, and/or bioerodable. Biostable refers to polymers that are not biodegradable. The terms biodegradable, bioabsorbable, and bioerodable, as well as degraded, eroded, and absorbed, are used interchangeably and refer to polymers that are capable of being completely eroded or absorbed when exposed to bodily fluids such as blood and can be gradually resorbed, absorbed and/or eliminated by the body. In certain embodiments, a suitable polymer herein is a fluid absorbing polymer. A fluid absorbing liquid polymer such as a hydrogel is infused into the interstices created within the three-dimensional braid structure and cured or fixed therein to create a composite structure in which the fluid absorbing polymer forms a continuous matrix and strong interface bonding with the fibers or strips Conning the reinforcement member. After the fluid absorbing polymer is cured or fixed, it can be dehydrated which reduces the volume of the implant. In this manner, the braided three-dimensional reinforcement member works in concert with the fluid absorbing-polymer since it is compressible. These properties allow the dehydrated implant to be shaped into an optimal cross-sectional configuration for implantation through a relatively small incision in an annulus. Reducing the size of the incision is advantageous since it reduces the area of trauma to the annulus and consequently decreases the available area for potential herniation and implant extrusion. It is also contemplated that substantially non-fluid absorbing elastic polymers can be utilized to form a polymeric matrix.

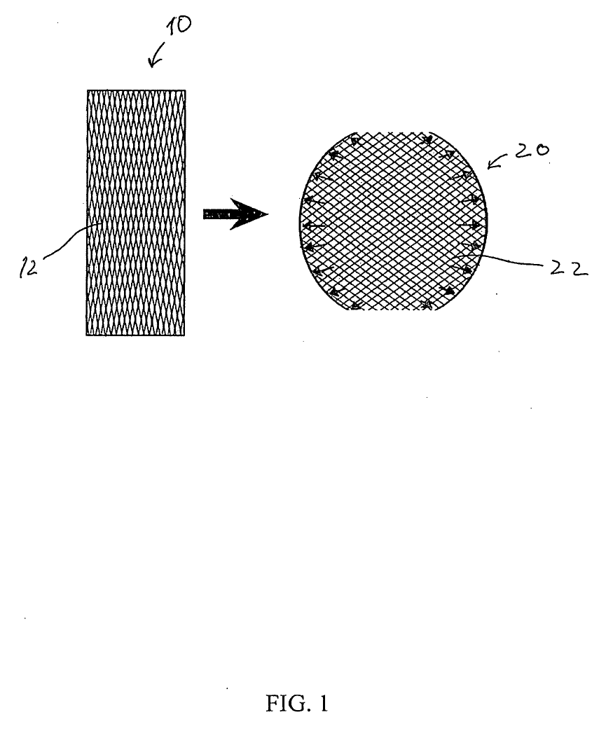

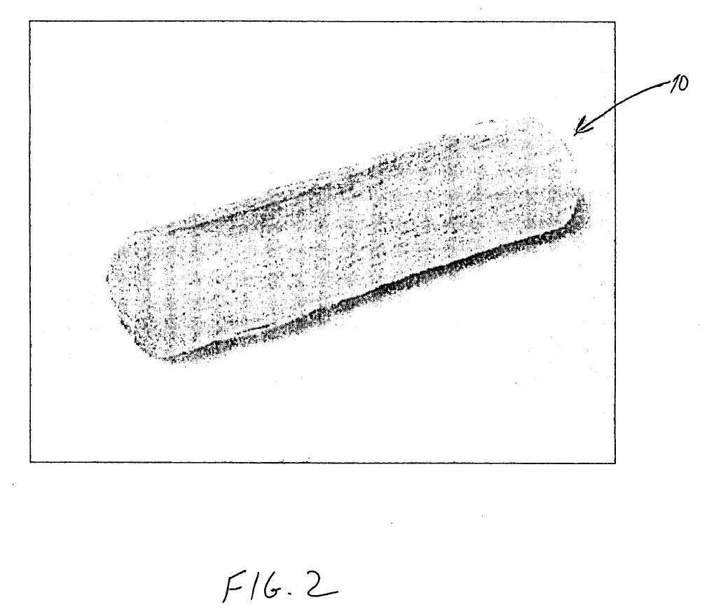

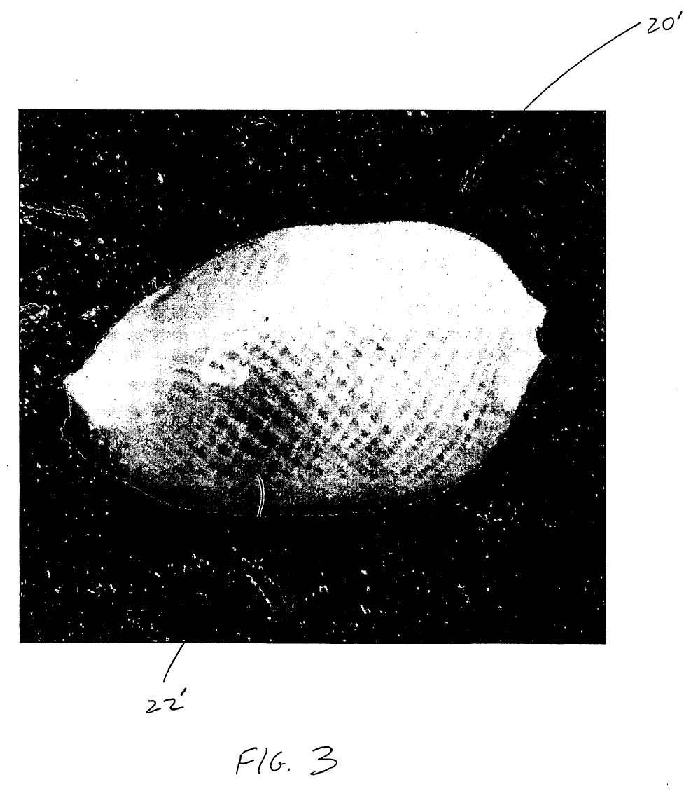

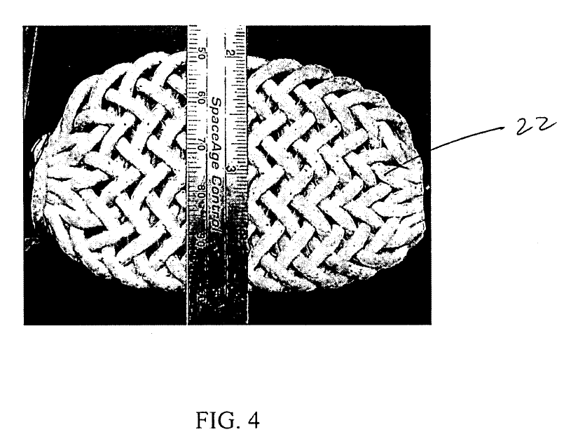

FIG. 1 schematically depicts an example of a compressed SNIC 10 having a three dimensional braided reinforcement member 12 embedded therein which transitions from a compressed configuration to and expanded disc-shaped configuration 20. In this case, the compressed SNIC 10 is in the shape of a cylinder. The three dimensional braided reinforcement member 22 is also seen to be in the expanded configuration. FIG. 2 is a perspective view of the compressed cylindrical SNIC 10. FIG. 3 is a perspective view of and expanded pillow-shaped SNIC 20′ having a three dimensional braided reinforcement member 22′ configured to correspond to the pillow shape of the SNIC 20′. An example of a pillow-shaped three dimensional braided reinforcement member 22′ is shown in top view in FIG. 4. The ruler is included to show that in this case, the flattened three dimensional braided reinforcement member is about 5 cm wide. When it assumes the pillow shape, the widest diameter of the three dimensional braided reinforcement member is less than about 5 cm.

A three dimensional braided reinforcement member of sufficient bulk is a robust structure able to withstand significant compressive, tensile and shear forces generated within the confines of the intervertebral space. Indeed, the textile structure alone provides an optimal backbone to the SNIC, due to its ability to resist complex loading while maintaining a very high amount of flexibility. This type of structure performs well when flexibility is required in a high strength composite while still allowing compression through suitable manufacturing processes which can generate higher compression forces than those generated in the body. A SNIC according to the present disclosure is advantageously dimensioned and configured to have a shape consistent with a cavity in an intervertebral space. As used herein, “a shape consistent with a cavity in an intervertebral space” means that the SNIC fits into a cavity located between adjacent vertebrae and complements at least a portion of the interior surface of the cavity. Thus, e.g., a SNIC may substantially fill the cavity or may fill only a portion of the cavity.

Fibers or strips useful for forming the three dimensional braided reinforcement member may be monofilament or multifilament or combinations of the two. Although the term “fiber” generally refers to a slender, elongated, threadlike object or structure of ellipsoid cross-section, for convenience, “fiber” as used herein also encompasses a “strip”, i.e., material which can be elongate and flat. Suitable materials and techniques for forming monofilament of multifilament fibers such as yarn or rovings are well-known to those skilled in the art. For example, suitable fiber forming materials include polyamide, polyethylene terephthalate, polypropylene, polyethylene, PEET, carbon, ceramic, glass and combinations thereof. Three-dimensional braiding techniques are also well-known to those skilled in the ail. See, e.g., Ko, Ceramic Bulletin, Vol. 68, No. 2, pp. 401-414 (1989). Advantageously, the fibers in a braid interlace at angles greater than zero, but less than ninety degrees. The orientation of the fibers or strips in a braid allows for three-dimensional malleability in a three-dimensional fiber architecture. In addition, the void to fiber ratio is adjustable, i.e., the architecture can be made more or less dense depending on the braiding angle and/or geometry of yam/roving cross-section. The void to fiber ratio can range from about 0.3 to about 3.0.

A particular advantage of three-dimensional braiding techniques is the ability to assume complex structural shapes. By utilizing an advantageous zigzag path within a three-dimensional architecture, the fibers are capable of shortening their length along defined dimensions and to elongate as well, until a desired jamming configuration is achieved. In one embodiment, the reinforcement member is configured and dimensioned to correspond to the shape of the void created by removal of the nucleus pulposus from the disc space. Accordingly, the reinforcement member may be configured in the shape of an ellipsoid cylinder to approximate the disc shape, e.g., ellipse, kidney or circular. Concave or convex structures are also contemplated. Alternatively, the three-dimensional reinforcement member may be configured into other geometric shapes such as rectangular, conical, frusto-conical or pyramidal. Irregular shapes may also be utilized. In certain embodiments, the reinforcement member can have a shape which does not correspond to the exterior shape of the SNIC. The reinforcement member may be hollow or filled with braided fiber. In addition, a braided reinforcement member may be engineered to be particularly conducive to anisotropic expansion and/or contraction, thus permitting a highly optimized delivery shape. Thus, the braided reinforcement member can be made to expand or be stretched along one axis while remaining relatively fixed along another axis. Such anisotropic expansion and contraction may be utilized to enhance preferential swelling of the SNIC in predetermined dimensions. In this manner, the reinforcement member enhances the ability of the SNIC to exert positive pressure against the vertebral end plates, thus mimicking the function of a natural nucleus pulposus which maintains the disc space between adjacent vertebrae while functioning as a shock absorber. By engineering the braided reinforcement member to resist radial expansion, outward and potentially damaging pressure on the annulus is minimized. Inherent anisotropic contraction may be facilitated by exerting sufficient pressure against the engineered contractile axis, thus allowing the braided reinforcement member to be selectively manipulated into a desired implantation shape of reduced and optimized cross-section.

The expandable polymeric matrix imparts further advantageous properties to the SNIC. For example, in expansion of the matrix, such as a fluid absorbing polymer, hydration of the polymer can cause the reinforcing member to assume larger dimensions than it would otherwise, e.g., in a relaxed state. The internal pressure created by the reinforcing member to counteract the expansion further stabilizes the outer dimension of the SNIC, thus helping to prevent deformation of the SNIC and extrusion from the disc space through the surgical incision site. In addition, the presence of fluid absorbing polymer allows the composite to respond to changing loads on the spine, similar to the native nucleus pulposus. The hydrogel encapsulates the textile fibers, thereby reducing or eliminating wear of the individual fibers, whether they are multifilament yams or monofilament as the composite experiences loading. Rather than fibers sliding past fibers, the motion between fibers is carried by the highly elastic fluid absorbing polymer. The amount of fluid absorbing polymer contained within the interstices of the braided three-dimensional reinforcement member can be varied based on the void to fiber ratio. The greater the void ratio, the more fluid absorbing polymer in the SNIC. Moreover, the fluid absorbing polymer provides a continuous, soft, lubricious surface to the anatomy rather than a porous semi-abrasive surface typical of textiles. In one embodiment, the outer dimensions of the braided reinforcement member are smaller than the outer dimensions of the SNIC, i.e., the braided reinforcement member is contained within the dimensions of the expandable polymeric matrix such that the polymer prevents the braided reinforcement member from contacting the vertebral end plates and annulus.

Suitable fluid absorbing polymers include synthetic polymers such as poly(ethylene glycol), poly(ethylene oxide), partially or fully hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose, and hydroxyalkylated celluloses such as hydroxyethyl cellulose and methylhydroxypropyl cellulose, and natural polymers such as polypeptides, polysaccharides or carbohydrates such as Ficoll™, polysucrose, hyaluronic acid, dextran, heparan sulfate, chondroitin sulfate, heparin, or alginate, and proteins such as gelatin, collagen, albumin, or ovalbumin or copolymers or blends thereof. As used herein, “celluloses” includes cellulose and derivatives of the types described above; “dextran” includes dextran and similar derivatives thereof. Examples of materials that can be used to form a hydrogel include modified alginates. Alginate is a carbohydrate polymer isolated from seaweed, which can be crosslinked to form a hydrogel by exposure to a divalent cation such as calcium. Alginate is ionically crosslinked in the presence of divalent cations, in water, at room temperature, to form a hydrogel matrix. Modified alginate derivatives may be synthesized which have an improved ability to form hydrogels.

Additionally, polysaccharides which gel by exposure to monovalent cations, including bacterial polysaccharides, such as gellan gum, and plant polysaccharides, such as carrageenans, may be crosslinked to form a hydrogel using methods analogous to those available for the crosslinking of alginates described above. Polysaccharides which gel in the presence of monovalent cations form hydrogels upon exposure, for example, to a solution comprising physiological levels of sodium. Hydrogel precursor solutions also may be osmotically adjusted with a nonion, such as mannitol, and then injected to form a gel.

Other polymeric hydrogel precursors include polyethylene oxide-polypropylene glycol block copolymers such as Pluronics™ or Tetronics™, which may be crosslinked by hydrogen bonding and/or by a temperature change. Other materials which may be utilized include proteins such as fibrin, collagen and gelatin. Polymer mixtures also may be utilized. For example, a mixture of polyethylene oxide and polyacrylic acid which gels by hydrogen bonding upon mixing may be utilized. In one embodiment, a mixture of a 5% w/w solution of polyacrylic acid with a 5% w/w polyethylene oxide (polyethylene glycol, polyoxyethylene) 100,000 can be combined to form a gel over the course of time, e.g., as quickly as within a few seconds.

Water soluble polymers with charged side groups may be crosslinked by reacting the polymer with an aqueous solution containing ions of the opposite charge, either cations if the polymer has acidic side groups or anions if the polymer has basic side groups. Examples of cations for cross-linking of the polymers with acidic side groups to form a hydrogel are monovalent cations such as sodium, divalent cations such as calcium, and multivalent cations such as copper, calcium, aluminum, magnesium, strontium, barium, and tin, and di-, tri- or tetra-functional organic cations such as alkylammonium salts. Aqueous solutions of the salts of these cations are added to the polymers to form soft, highly swollen hydrogels and membranes. The higher the concentration of cation, or the higher the valence, the greater the degree of cross-linking of the polymer. Additionally, the polymers may be crosslinked enzymatically, e.g., fibrin with thrombin.

Suitable ionically crosslinkable groups include phenols, amines, imines, amides, carboxylic acids, sulfonic acids and phosphate groups. Aliphatic hydroxy groups are not considered to be reactive groups for the chemistry disclosed herein. Negatively charged groups, such as carboxylate, sulfonate and phosphate ions, can be crosslinked with cations such as calcium ions. The crosslinking of alginate with calcium ions is an example of this type of ionic crosslinking. Positively charged groups, such as ammonium ions, can be crosslinked with negatively charged ions such as carboxylate, sulfonate and phosphate ions. Preferably, the negatively charged ions contain more than one carboxylate, sulfonate or phosphate group.

Anions for cross-linking of the polymers to form a hydrogel are monovalent, divalent or trivalent anions such as low molecular weight dicarboxylic acids, for example, terepthalic acid, sulfate ions and carbonate ions. Aqueous solutions of the salts of these anions are added to the polymers to form soft, highly swollen hydrogels and membranes, as described with respect to cations.

A variety of polycations can be used to complex and thereby stabilize the polymer hydrogel into a semi-permeable surface membrane. Examples of materials that can be used include polymers having basic reactive groups such as amine or inline groups, having a preferred molecular weight between 3,000 and 100,000, such as polyethylenimine and polylysine. These are commercially available. One polycation is poly(L-lysine); examples of synthetic polyamines are: polyethyleneimine, poly(vinylamine), and poly(allyl amine). There are also natural polycations such as the polysaccharide, chitosan.

Many hydrogel polymers can be deformed, frozen into a deformed shape and they can maintain that shape indefinitely or until, e.g., a temperature change causes the polymer to “relax” into the shape originally held prior to freezing. This property may be referred to as shape memory or frozen deformation by those skilled in the art.

The temperature at which frozen deformation occurs is referred to as the glass transition temperature or T_{g}. At T_{g} several polymer properties such as density, entropy and elasticity may sharply change. Many polymers can be mixed with agents that can have a drastic effect on a polymer T_{g}. Polymers which absorb fluid are of particular interest and water is the preferred T_{g} altering agent. Hydrogels which contain less than about five percent water may be considered dehydrated or xerogels. The T_{g} of a xerogel will change as it absorbs fluids containing water. Once the T_{g} becomes lower than ambient the now partially hydrated hydrogel becomes pliant and may be elastically deformed. If the polymer is held in a state of elastic deformation while the T_{g} is raised above ambient the polymer will maintain the deformed state indefinitely. This can be accomplished by either lowering the ambient temperature (freezing) or by returning the polymer to its xerogel state thus raising the T_{g}.

Using this method, hydrogel articles may be produced with vastly differing xerogel shapes compared to hydrated shapes. This is especially useful in cases such as medical implants where, in delivering a prosthesis into the human body, every care should be taken to reduce trauma to the patient. For example, a SNIC which is shaped as an ellipsoidal cylindrical disc, for instance, may be re-shaped, in accordance with the present invention, into a tapered elongate rod in order to facilitate minimally invasive implantation. Once the implant is indwelling and has absorbed water containing fluids, it will substantially return to the shape of the disc and maintain that shape indefinitely. As used herein, “substantially” is intended to mean any of “approximately”, “nearly” or “precisely.”

A preferred polymer configuration includes two polymer phases of different hydrophilicity, the less hydrophilic phase having higher content of hydrophobic groups and more hydrophilic phase having higher content of hydrophilic groups. The less hydrophilic phase is preferably crystalline and more hydrophilic phase is preferably amorphous, as can be established from X-ray diffraction.

Advantageous hydrophobic groups are pendant nitrile substituents in 1,3 positions on a polymethylene backbone, such as poly(acrylonitrile) or poly(methacrylonitrile). The hydrophilic phase may preferably contain a high concentration of ionic groups. Preferred hydrophilic groups are derivatives of acrylic acid and/or methacrylic acid including salts, acrylamidine, N-substituted acrylamidine, acrylamide and N-substituted acryl amide, as well as various combinations thereof. A particularly preferred combination contains approximately two thirds acrylic acid and its salts (on molar basis), the rest being a combination of plain and N-substituted acrylamides and acrylamidines.

At least one polymeric component is preferably a multiblock copolymer with alternating sequences of hydrophilic and hydrophobic groups. Such sequences are usually capable of separating into two polymer phases and form strong physically crosslinked hydrogels. Such multiblock copolymers can be, for example, products of hydrolysis or aminolysis of polyacrylonitrile or polymethacrylonitrile and copolymers thereof. For convenience, polymers and copolymers having at least about 80 molar % of acrylonitrile and/or methacrylonitrile units in their composition may be referred to as “PAN”. Hydrolysis and aminolysis of PAN and products thereof are described, for example, in U.S. Pat. Nos. 4,107,121; 4,331,783; 4,337,327; 4,369,294; 4,370,451; 4,379,874; 4,420,589; 4,943,618 , and 5,252,692, each being incorporated herein by reference in their respective entireties.

A preferred fluid absorbing polymer for the SNIC is a synthetic composite of a cellular (or domain) type with continuous phase formed by a hydrophobic polymer or a hydrophilic polymer with low to medium water content forming a “closed cell” spongy structure that provides a composite with good strength and shape stability. Examples of suitable polymers are polyurethanes, polyureas, PAN, polydimethylsiloxanes (silicone rubber), and highly crystalline multiblock acrylic and methacrylic copolymers. The polymer should be sufficiently permeable to water. It is known that even distinctly hydrophobic polymers, such as silicone rubber, can form swellable composites. More preferably, the continuous phase is formed by a strong hydrophilic polymer with sufficient permeability for water but impermeable to high-molecular solutes. Examples of such polymers are highly crystalline hydrogels based on segmented polyurethanes, polyvinylalcohol or multiblock acrylonitrile copolymers with derivatives of acrylic acid. Typically, suitable polymers for the continuous phase in cellular composites have a water content in fully hydrated state between about 60% by weight and about 90% by weight, preferably between about 70% and about 85% by weight.

The second component of the fluid absorbing polymer may be a highly hydrophilic polymer of high enough molecular weight to prevent permeation of the hydrophilic polymer through the continuous phase. This component is contained inside the matrix of the continuous phase. The entrapped hydrophilic polymers (the so-called “soil block”) may be high-molecular weight water-soluble polymers, associative water-soluble polymers or highly swellable hydrogels containing, in a fully hydrated state, an amount of hydration which is preferably at least about 5%. greater than the hydrophobic component. For example, the second component hydrated to at least about 65% when the first component is hydrated to about 60%. In other embodiments, e.g., from the second component could be fully hydrated at from about 95% of water and up to about 99.8% of water. Such hydrogels are very weak mechanically. However, it may not matter in composites where such polymers' role is generation of osmotic pressure rather than load-bearing, with e.g., compression strength in full hydration in the range of about 0.01 MN/m² or lower.

A system with closed cells (or domains) containing highly swellable or water-soluble polymers can form composites with very high swelling pressure as needed for the SNIC function. Examples of suitable hydrophilic polymers are high-molecular weight polyacrylamide, polyacrylic acid, polyvinylpyrrolidone, polyethyleneoxide, copolymers of ethyleneoxide and propyleneoxide or hyaluronic acid; covalently crosslinked hydrogels such as hydrophilic esters or amides of polyacrylic or polymethacrylic acids; and physically crosslinked hydrogels, such as hydrolyzates or aminolysates of PAN.

Particularly suitable are associative water-soluble polymers capable of forming very highly viscous solutions or even soft physical gels. Preferred are associative polymers containing negatively charged groups, such as carboxylates, sulpho-groups, phosphate groups or sulfate groups. Particularly preferred are associative polymers formed by hydrolysis and/or aminolysis of PAN to high but finite conversions that leave a certain number of nitrile groups (typically, between about 5 and 25 molar %) unreacted.

Preferred fluid absorbing polymer composites have both a continuous phase and a dispersed phase formed by different products of hydrolysis or aminolysis of PAN. In this case, both components are compatible and their hydrophobic blocks can participate in the same crystalline domains. This improves anchorage of the more hydrophilic component and prevents its extraction or disassociation. The size of more hydrophilic domains may vary widely, from nanometers to millimeters, preferably from tens of nanometers to microns.

The ratio between the continuous discrete phase (i.e., between more hydrophobic and more hydrophilic components may vary from about 1:2 to about 1:100 on a dry weight basis, and a preferred ratio ranges from about 1:5 to about 1:20. Examples of compositions and implants are described in U.S. Pat. Nos. 6,264,695 and 6,726,721, both of which are incorporated herein by reference in their entireties. A preferred method of making the fluid absorbing polymer composite is described in U.S. Pat. No. 6,232,406, herein incorporated by reference in its entirety.

Examples of particularly suitable hydrogel forming copolymers are prepared by a partial alkaline hydrolysis of polyacrylonitrile (“HPAN”) in the presence of sodium thiocyanate (NaSCN). The resulting hydrolysis product is a multi-block acrylic copolymer, containing alternating hydrophilic and hydrophobic blocks. Hydrophilic blocks contain acrylic acid, acrylamidine, and acrylamide. In one embodiment, for example, a PAN hydrolysate polymer (referred to herein HPAN I) (46±1% conversion of hydrolysis) having the following composition: acrylonitrile units ˜53-55%, acrylic acid units ˜22-24%, acrylamide units ˜17-19%, acrylamidine units ˜4-6%, as determined by ¹³C NMR, is dissolved in a suitable solvent such as a ˜55% solution of sodium thiocyanate in water to form a viscous solution. The viscous solution is poured into a porous mold having, e.g., a cylindrical shape. The solution can then be solvent cast, e.g., by solvent exchange (e.g., water for NaSCN). The pores should be sufficiently small as to not permit the polymer to diffuse or leak out of the mold.

A more rigid fluid absorbing polymer may be another PAN hydrosylate polymer, referred to herein as HPAN II (28±1% conversion of hydrolysis), having the following composition: acrylonitrile units ˜71-73%, acrylic acid units ˜13-15%, acrylamide units ˜10-12%, acrylamidine units ˜2-4%, as determined by ¹³C NMR, disolved in ˜55% NaSCN which can be solvent cast, washed, dried and cut to a suitable shape.

Representative examples of other polymers that may be used to fabricate a polymeric matrix for SNICs herein include, but are not limited to, poly(N-acetylglucosamine) (Chitin), Chitosan, poly(3-hydroxyvalerate), poly(lactide-co-glycolide), poly(3-hydroxybutyrate), poly(4-hydroxybutyrate), poly(3-hydroxybutyrate-co-3-hydroxyvalcrate), polyorthoester, polyanhydride, poly(glycolic acid), poly(glycolide), poly(L-lactic acid), poly(L-lactide), poly(D,L-lactic acid), poly(D,L-lactide), poly(L-lactide-co-D,L-lactide), poly(caprolactone), poly(L-lactide-co-caprolactone), poly(D,L-lactide-co-caprolactone), poly(glycolide-co-caprolactone), poly(trimethylene carbonate), polyester amide, poly(glycolic acid-co-trimethylene carbonate), co-poly(ether-esters) (e.g. PEO/PLA), polyphosphazenes, biomolecules (such as fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose. Additional representative examples of polymers that may be useful in fabricating embodiments of SNICs disclosed herein include ethylene vinyl alcohol copolymer (commonly known by the generic name EVOH or by the trade name EVAL), poly(butyl methacrylate), poly(vinylidene fluoride-co-hexafluoropropene) (e.g., SOLEF 21508, available from Solvay Solexis PVDF, Thorofare, N.J.), polyvinylidene fluoride (otherwise known as KYNAR, available from ATOFINA Chemicals, Philadelphia, Pa.), ethylene-vinyl acetate copolymers, poly(vinyl acetate), styrene-isobutylene-styrene triblock copolymers, and polyethylene glycol.

A SNIC may be manufactured by providing a three-dimensional braided reinforcement member of desired configuration and placing it in a mold. A fluid polymer such as a fluid absorbing liquid polymer is added to the mold and infuses into the interstices of the three-dimensional braid until the reinforcement member is preferably saturated. In one embodiment, a gap, e.g., about 1 mm, is left between one or more sides of the three-dimensional braided reinforcement member and the walls of the mold. Fluid absorbing liquid polymer is allowed to fill the gap between the mold and the reinforcement member. As the reinforcement member absorbs fluid absorbing liquid polymer additional amounts of the fluid absorbing liquid polymer can be added. When the fluid absorbing polymer is cured or fixed, e.g., by solvent casting, ionic gelation, photo-polymerization and the like, it solidifies and creates a continuous matrix throughout the reinforcement member and also forms a layer surrounding and encapsulating the reinforcement member. In the case of solvent casting, the mold may be made of material which is impermeable to the fluid absorbing polymer but permeable to water. The mold is placed in a water bath to extract the solvent (e.g., sodium thiocyanate) which causes the polymer to coagulate. The mold may then be opened and any remaining solvent in the SNIC is extracted. If it is desired to leave one or more sides of the SNIC open to the reinforcement member, then the desired side(s) of the reinforcement member is placed up against the wall of the mold to prevent formation of a gap for the liquid fluid absorbing polymer to fill.

In one embodiment, the fluid absorbing polymer is made to achieve a strong physical bond to the fibers of the braided three-dimensional reinforcement member by incorporating an initial treatment of the fibers of the member, either before or after the braiding process, with a relatively hydrophobic fluid absorbing polymer to create an encapsulating layer of the relatively hydrophobic fluid absorbing polymer. For example, a hydrogel such as HPAN II is applied to the fibers of the braid as a 10% solution by weight in a solvent (sodium thiocyanate 55% by weight in water) and then coagulated onto the fibers by solvent exchange with an aqueous solution such as water. As the polymer coagulates, it shrinks volumetrically around the fibers, causing a tight physical bond to the fibers. If desired, the treated braided three-dimensional reinforcement member is placed in a mold and a relatively more hydrophilic fluid absorbing polymer in the liquid state is added to create a cohesive continuous polymer matrix which surrounds the braided three-dimensional reinforcement member. For example, a 10% by weight HPAN I in a 55% by weight sodium thiocyanate solution, is added to the mold. The solvent from the HPAN I solution causes the outermost surface of the coagulated HPAN II layer surrounding the braided fibers to dissolve and allow commingling of the HPAN I and HPAN II hydrogel polymers at the surface interface which forms a strong adhesive bond when the HPAN I and commingled hydrogels are coagulated by solvent exchange.

It is contemplated that regions of more or less modulus of elasticity and durability may be incorporated into the SNIC. For example, it may be desirable to place a relatively more rigid fluid absorbing polymer at the top and bottom of the SNIC, i.e., the portions which contact the vertebral end plates. Accordingly, a liquid fluid absorbing polymer such as HPAN II can initially be added to the mold to create a first layer, followed by placement of the reinforcing member into the liquid polymer such that the polymer covers and is absorbed into the bottom, e.g., one-third of the reinforcement member. Increasing air pressure can speed the process of saturation of the implant. After a sufficient amount of liquid polymer is absorbed, it can be cured or fixed. If a softer layer of fluid absorbing polymer is desired in the center section of the SNIC, a hydrogel such as HPAN I can be added over the bottom layer to fill the mold to, e.g., ⅔ capacity. After the HPAN I is absorbed sufficiently into the reinforcement member, it can be cured or fixed to create a relatively soft middle layer. A third, more rigid layer can then be created by filling the rest of the mold with, e.g., HPAN II and curing or fixing it by solvent casting. It should be understood that any number of layers of varying or the same thickness may created in this fashion. In addition, different fluid absorbing polymers can be used to create zones with different properties. If desired, an adhesive can be added between adjacent layers to insure bonding or, e.g., in the case of the HPAN polymers, the layers can be made to naturally adhere to one another. In one embodiment, one or more layers of liquid fluid absorbing polymer can be placed on top of other liquid layers of fluid absorbing polymer and then cured. Differences in density keep the layers from completely intermixing. Some co-mingling of liquid fluid absorbing polymers at layer interfaces can provide for an advantageous smooth transition between layers and reduce or eliminate the need for an adhesive between layers.

The order of layering may be varied to suit particular applications. After the last layer is applied, the mold is closed and placed in water for solvent exchange in the case of HPAN I and/or HPAN II. For example, a sodium thiocyanate solution diffuses out and is replaced with water, causing the viscous solution to coagulate. In the case of successive layers of HPAN I and HPAN II, the layers adhere to each other without the need for any adhesives. As mentioned above, in certain embodiments, the interface between the HPAN I layers and the HPAN II layers is blurred by comingling of the polymers during the manufacturing process, leading to a gradual transition from layer to layer. In other embodiments, adhesives such as polyurethanes or cyanoacrylates are used to bond the layers together.

In another embodiment, a three-dimensional reinforcement member has a hollow core which can be filled with a fluid absorbing polymer of relatively high or low modulus of elasticity as compared to an exterior layer of fluid absorbing polymer. For example, a hollow cylindrical three-dimensional-reinforcement member is filled with HPAN I and then surrounded by a layer of HPAN II. As with radially stacked layers, the two polymers can be made to intermix at the the boundary between them or an adhesive can be used. It is contemplated that layers of hydrogel polymers can be used with elastic non-hydrogel polymers in both the radially stacked layer embodiments and in the hollow core tilled embodiments. As indicated above, a myriad of geometric shapes can be utilized in connection with the three-dimensional reinforcement member. In one embodiment, a cylindrical hollow core three-dimensional reinforcement member has a “U.S. football” shape, i.e., a cylindrical shape having tapered ends, with a maximum diameter located in the central portion, e.g., of about 12 mm and a longitudinal axis of about 34 mm. The walls of the three-dimensional reinforcement member are, e.g., about 5 mm thick. It should be understood that these dimensions are exemplary and that they can be varied to suit particular needs. When incorporated into a SNIC, the bulging central portion of a “football” shape advantageously fits into the opposing concave portions of adjacent vertebral endplates. The “football” shape can be achieved by providing a braided tube, heat sealing the ends and pushing the ends inwardly toward the center, thus causing the central portion to bulge outwardly. The three-dimensional reinforcement member is placed in a mold and filled with a 10% by weight HPAN I in sodium thiocyanate solution (55% by weight) which has been heated to a temperature of about 50° C. for about 18 hours to facilitate infusion of the fluid absorbing polymer. The fluid absorbing polymer is then coagulated, e.g., by solvent exchange (e.g., water for NaSCN). The coagulated fluid absorbing polymer will maintain the football shape of the three-dimensional reinforcement member. The filled three-dimensional reinforcement member is then placed in a cylindrical mold with walls that are 2 mm wider and higher than the dimensions of the three-dimensional reinforcement member. The mold is filled with HPAN II solution and then coagulated by solvent extraction in an aqueous solution. It should be understood that the percentage of HPAN in solution can be varied based on the molecular weight of the HPAN and the degree of desired viscosity. For example, HPAN with a molecular weight of about 240.000 daltons will form a relatively viscous solution at lower concentrations, e.g., about 5% to about 11% by weight, more preferably about 9%. As compared to HPAN having a molecular weight of about 240,000 daltons, HPAN with a molecular weight of about 85,000 daltons is relatively less viscous in solution and can be utilized in concentrations ranging, e.g., from about 5% to about 25% by weight. Temperature of the solution can vary from about room temperature to about 80° C., but is preferably about 50° C. The infusion or soaking time can be varied as well, e.g., from about 3 to 24 hours, or more, as long as the fluid absorbing polymer is adequately incorporated in the interstices of the braided structure and fills the core of the member.

In another embodiment, a process for making a SNIC involves utilizing a fluid absorbing polymer insert for insertion into the cavity of the hollow core of the three-dimensional reinforcement member. The insert is produced by fashioning the fluid absorbing polymer into a predetermined shape which corresponds to the desired shape of the cavity of the hollow braid when the insert would be in a substantially fully hydrated state. The insert can be made by filling a mold of predetermined dimension with the fluid absorbing polymer in a liquid state and causing it to solidify or coagulate. Alternatively, the insert can be heat shaped into the desired dimensional configuration. This interior cast, made, e.g., of a hydrogel such as HPAN I, is then inserted into a tubular three-dimensional braid. Using the football shape as an example, the football-shaped insert is inserted into an open end of the tubular braid. The ends of the braid are heat sealed using a hot iron or knife. The braided three-dimensional reinforcement member containing the insert is then placed into a mold large enough to accommodate it plus one millimeter of free space on all exterior surfaces. Upon filling this mold with a 9% by weight solution of HPAN II (MW 240,000) in sodium thiocyanate solution, and coagulating the assembly using an aqueous solution, the hydrogel polymers strongly interact (solvent bonding), resulting in a device with a soft, high expansion ratio gel interior and a tough outer hydrogel jacket that is tightly integrated with the braided three-dimensional reinforcement member.

In another embodiment, a polymeric matrix from a substantially non-fluid absorbing elastic polymer is utilized to create the continuous matrix which is reinforced by the braided three-dimensional reinforcement member. For example, a substantially non-fluid absorbing elastic polymer includes biocompatible in-situ curable elastic polymers such as polyurethane, silicone, and the like. It should be understood that the substantially non-fluid absorbing elastic polymer can absorb small amounts of fluid in some interstitial spaces. Accordingly, liquid precursors of such polymers can be used in place of or in addition to the fluid absorbing polymers described herein. It is also contemplated that the polymeric matrix made of a biocompatible in-situ curable elastic polymer can be cured or otherwise solidified in vivo. Advantageously, a preformed braided three-dimensional reinforcement member having a hollow core can be inserted into an intervertebral disc space in a collapsed configuration and the curable liquid polymer is infused into the hollow core, thus causing the braided three-dimensional reinforcement member to expand in size into a predetermined shape defined by the braiding process. In one embodiment, after expansion to a desired dimension, additional curable liquid polymer is added to surround the reinforcement member in the disc space, thus preventing contact of the braided three-dimensional reinforcement member with the interior surfaces of the intervertebral space. In another embodiment, infusion of the curable liquid polymer into the core is continued such that after the braided three-dimensional reinforcement member has expanded to its maximum dimension, the liquid polymer is forced through the interstices of the braid to form a layer of polymer on the exterior surface of the SNIC. After filling to a desired volume, the polymer is then cured.

In another embodiment, one or more tethers such as a string, suture, etc., is incorporated into the SNIC, preferably in a central location. The tether may be utilized in positioning or maintaining the position of the SNIC, or its components, during manufacture in molds, and after manufacture as a device for positioning the SNIC within an intervertebral space. The tether may be simply placed in a central location within the hollow cavity of a braided three-dimensional reinforcement member prior to filling with a liquid fluid absorbing polymer and is then present when the cavity is filled. Alternatively, a tether may be incorporated into the center of a mold when a liquid fluid absorbing polymer insert is coagulated. Alternatively, the tether may be attached directly to the braided three-dimensional reinforcement member.

In certain embodiments, upon completion of the solvent exchange extraction process the SNIC is hydrated to its fullest extent (˜90% equilibrium water content (EWC)). In this fully hydrated state the SNIC is readily deformed under modest loads and the hydrogel, e.g., HPAN I OR HPAN II, glass transition temperature (T_{g}) is well below room temperature. This is the “relaxed” state of the SNI, the state to which it will return after loading below the critical level. The critical level is the point at which permanent deformation occurs and is further discussed below. In accordance with the present invention, the fully hydrated SNIC is deformed into a desirable second shape and the temperature of the SNI is lowered below its T_{g} (near freezing point of water). Such a SNIC would be said to be in a state of “frozen deformation” and it would retain that deformed shape indefinitely. Once the SNIC is warmed above its T_{g}, however, the SNIC would recover to it's original memorized configuration.

The T_{g} of the hydrogel increases with decreasing water content. This characteristic is exploited by simultaneously raising the T_{g} while deforming the SNIC into a desired shape. In other words, as the SNIC dehydrates it is freezing the position of the polymer chains. To regain the original shape of the SNIC, the T_{g} may be lowered by hydration.

In order to obtain a preferred rod-shape having an optimal cross-sectional ellipsoid shape for implantation, e.g., suppository, bullet, tapered cylinder, etc., from, e.g., an ellipsoid cylindrical SNIC, deformation is advantageously maintained radially, substantially parallel with the major elliptical axis or diameter. This is accomplished by placing the implant within a radially collapsible member for exerting substantially equilateral circumferential compression on an object, e.g., an SNIC, contained within the member. Suitable radially collapsible members include, e.g., a flexible sleeve such as a braided sock, a flexible coil, iris diaphragm, collapsible loop, etc. In a preferred embodiment, the radially collapsible member is porous or semipermeable so that water, either as liquid or as vapor, passes through the member. The collapsible member may be made of an elastic material such as rubber or neoprene fabric which has been made porous by any technique known to those skilled in the art, or a woven or non-woven mesh or braid. The collapsible member may also be made of a flexible, metal having sufficient porosity to allow water to exit from the implant. The collapsible member does, however, need to be stiff enough to be able to exert sufficient compressive force when tension is applied to compress the SNIC, i.e., it should not be so elastic that it deforms without being able to exert sufficient compressive force.

In operation, the radially collapsible member exerts substantially equilateral circumferential compression on the implant by substantially uniformly decreasing in diameter while contacting the implant. The preferred porous nature of the collapsible member allows water from the implant to escape into the surrounding environment so that the implant can become dehydrated. In one embodiment, the sleeve radially collapsible member is stretched in length which causes the inner diameter to decrease, thus compressing the SNIC, including the braided three-dimensional reinforcement member, into a desired implantation configuration. A more complete description of a suitable radial compression process is described in U.S. application Ser. No. 11/303,767, herein incorporated by reference in its entirety. Other methods of reducing the profile of the SNIC include folding or rolling the SNIC into, e.g., bellows, a taco shape or a cigar shape.

A SNIC according to the disclosure herein may contain a medicinal agent. “Medicinal agent” is used in its broadest sense and it includes any substance or mixture of substances which may have any clinical use. It is to be understood that medicinal agent encompasses any drug, including hormones, antibodies, therapeutic peptides, etc., or a diagnostic agent such as a releasable dye which has no biological activity per se. Thus, in its broadest aspect, a method of delivery herein may be defined as the release of any substance for clinical use, which may or may not exhibit biological activity.

Examples of medicinal agents that can be used include anticancer agents (cisplatin, tamoxifen, etc.), analgesics (opioids, acetaminophen, etc.), anesthetics (lidocaine, benzodiapines, etc), anti-inflammatory agents (prednisolone, ibuprofen, etc), therapeutic proteins, antimicrobials, demineralized bone and radiopaque materials. Such medicinal agents are well-known to those skilled in the art. For example, therapeutic proteins include growth factors. Therapeutic proteins include bone morphogenetic proteins (BMPs) such as BMP1, BMP2, BMP3, BMP4, BMP5, BMP6, BMP7, and BMP8a. Agents such as demineralized bone and growth factors provide a modality to encourage ingowth of bone into the SNIC when desired. The medicinal agents may be in the form of dry substance in aqueous solution, in alcoholic solution or particles, microcrystals, microspheres or liposomes. An extensive recitation of various medicinal agents is disclosed in Goodman and Gilman, The Pharmacological Basis of Therapeutics, 10th ed. 2001, or Remington, The Science and Practice of Pharmacy, 21 ed. (2005). As used herein, the term “antimicrobial” is meant to encompass any pharmaceutically acceptable agent which is substantially toxic to a pathogen. Accordingly, “antimicrobial” includes antiseptics, antibacterials, antibiotics, antivirals, antifungals and the like. Radiopaque materials include releasable and non-releasable agents which render the SNIC visible in any known imaging technique such as X-ray radiographs, magnetic resonance imaging, computer assisted tomography and the like. The radiopaque material may be any conventional radiopaque material known in the art for allowing radiographic visualization of an implant, and may be, e.g., metal wire or Hakes made from a biocompatible material, such as titanium, tantalum, stainless steel, or nitinol; or metallic salts (such as barium compounds).

Medicinal agents may be incorporated into the SNIC at various points in the manufacturing process. For example, a suitable medicinal agent can be mixed with a polymer such as a fluid absorbing liquid polymer before it is cured or fixed. Alternatively, a suitable medicinal agent may be dissolved into a solvent cast solution and then diffused into the hydrogel in accordance with normal kinetic principles. If the SNIC is then dehydrated, the medicinal agent collects in the interstices of the hydrogel and/or the braided three-dimensional reinforcement member.

A dehydrated SNIC according to the disclosure herein maybe sterilized by any suitable conventional means, e.g., ethylene oxide, irradiation, etc. and packaged for distribution. A kit containing the sterilized SNIC and a package insert describing the SNIC, along with instructions is useful for medical practitioners. The SNIC may be implanted posteriorly or anteriorly, depending on the indication, into the disc space by making a small incision in the annulus which corresponds in size to the radial axis length (or less, e.g., one half radial axis length) of the SNIC. In a preferred embodiment, an incision is made in the annulus which is less than the cross-sectional diameter of the dehydrated implant, e.g., approximately one-half the diameter of a dehydrated rod-shaped implant. A blunted or tapered end of the implant is inserted into the incision and the implant itself serves as a device for dilating the incision to substantially the cross-sectional dimensions of the SNIC. In this manner, the length of the incision is minimized and efficiently sized directly to the implant cross-section. After passing through the annulus, the SNIC is inserted into the disc space where it hydrates by absorption of fluid present in the disc space. Thus, the dehydrated SNIC can have a first configuration with a reduced or smaller cross-section to optimize insertion through an incision which transisitions to a second configuration of expanded dimension achieved by hydration of the SNIC. In a preferred embodiment, the SNIC is configured to expand (when it is not constrained by the end plates of the adjacent vertebrae) to a height which is greater than the disc space by about 5% to about 20% or greater. It is contemplated that, in one embodiment, a boomerang-shaped SNIC can be inserted which advantageously facilitates positioning of the SNIC within the disc space by providing a SNIC shape which partially conforms to the curved interior of the disc space. After insertion, the annulus may be sealed by any means known to those skilled in the art and, as it is hydrated by body fluids, the SNIC expands to substantially fill the void in the disc space.

It should be understood that the examples and embodiments of the invention provided herein are preferred embodiments. Various modifications may be made to these examples and embodiments without departing from the scope of the invention. For example, those skilled in the art may envision different braiding techniques used to create the braided three-dimensional reinforcement member. Other three-dimensional shapes which are capable of reinforcing a SNIC other than those specifically discussed herein may be utilized. In addition, those skilled in the art may envision additional polymers and/or hydrogels which can be dehydrated and shaped according to the techniques described herein. Similarly, the shapes of the hydrated SNICs described herein are exemplary and any suitable hydrated SNIC shape can be dehydrated to create an optimally shaped, substantially dehydrated SNIC for minimally invasive insertion into the disc space. In addition, process parameters such as temperature, humidity, pressure, time and concentration may be varied according to conventional techniques by those skilled in the art to optimize, results.

What is claimed is:

1. A spinal nucleus implant comprising a braided three-dimensional reinforcement member having a polymeric matrix imbued therein, the implant configured to have a shape consistent with a cavity within an intervertebral disc space.

2. A spinal nucleus implant according to claim 1 wherein the polymeric matrix is made of a fluid absorbing polymer.

3. A spinal nucleus implant according to claim 2 wherein the polymer is a hydrogel.

4. A spinal nucleus implant according to claim 3 wherein the hydrogel is selected from the group consisting of polyacrylonitrile, polyvinyl alcohol, polyacrylamide, silicone, polyurethane, poly(ethylene glycol), poly(ethylene oxide)-poly(propylene oxide) copolymer, polymethacrylate, polyhydroxyethylmethacrylate, hydroxyethylmethacrylate-methylmethacrylate copolymer, polyvinylpyrrolidone, hyaluronic acid, chondroitin sulfate, carboxymethyl cellulose, hydroxypropylmethyl cellulose, collagen, fibrin, alginate, and agar.

5. A spinal nucleus implant according to claim 1 wherein the braided three-dimensional reinforcement member is made of a fiber selected from the group consisting of monofilament, multifilament and combinations thereof.

6. A spinal nucleus implant according to claim 1 wherein the braided three-dimensional reinforcement member is made of a fiber made from a material selected from the group consisting of polyamide, polyethylene terephthalate, polypropylene, polyethylene, PEET, carbon, ceramic, glass and combinations thereof.

7. A spinal nucleus implant according to claim 1 wherein the braided three-dimensional reinforcement member is configured to correspond to the exterior shape of the implant.

8. A spinal nucleus implant according to claim 7 wherein wherein the braided three-dimensional reinforcement member is configured in the shape of a cylinder having an enlarged central portion and tapered ends.

9. A spinal nucleus implant according to claim 1 wherein the braided three-dimensional reinforcement member is encapsulated by a further polymeric matrix which may be the same or different than the polymeric matrix imbued therein.

10. A spinal nucleus implant according to claim 1 wherein the void ratio in the braided three-dimensional reinforcement member ranges from about 0.3 to about 3.0.

11. A spinal nucleus implant according to claim 1 further comprising a medicinal agent.

12. A spinal nucleus implant according to claim 1 wherein the braided three-dimensional reinforcement member defines a core which contains at least a portion of the polymeric matrix.

13. A spinal nucleus implant according to claim 1 wherein the implant is configured to transform from a first configuration to a second configuration, the first configuration having a smaller cross-section than the second configuration.

14. A spinal nucleus implant according to claim 13 wherein the first configuration is bullet shaped and the second configuration is an expanded cylindrical ellipsoid configured to substantially fill an intervertebral disc space and exert positive pressure against the vertebral endplates which define opposing surfaces of the disc space.

15. A spinal nucleus implant according to claim 1 wherein the polymeric matrix is made of a substantially non-fluid absorbing in-situ curable elastic polymer.

16. A spinal nucleus implant according to claim 1 further comprising a medicinal agent.

17. A method of making a spinal nucleus implant comprising providing a braided three-dimensional reinforcement member configured and dimensioned to have a shape consistent with a cavity in an intervertebral space and infusing the braided three-dimensional reinforcement member with a liquid precursor of a polymeric matrix.

18. A method of making a spinal nucleus implant according to claim 17 wherein the polymeric matrix if formed from a fluid absorbing polymer.

19. A method of making a spinal nucleus implant according to claim 18 wherein the fluid absorbing polymer is selected from the group consisting of polyacrylonitrile, polyvinyl alcohol, polyacrylamide, silicone, polyurethane, poly(ethylene glycol), poly(ethylene oxide)-poly(propylene oxide) copolymer, polymethacrylate, polyhydroxyethylmethacrylate, hydroxyethylmethacrylate-methyhnethacrylate copolymer, polyvinylpyrrolidone, hyaluronic acid, chondroitin sulfate, carboxymethyl cellulose, hydroxypropylmethyl cellulose, collagen, fibrin, alginate, and agar.

20. A method of making a spinal nucleus implant according to claim 17 wherein the braided three-dimensional reinforcement member is placed within a mold cavity which has dimensions greater than the braided three-dimensional reinforcement member to allow the fluid absorbing polymer to be absorbed into and saturate the braided three-dimensional reinforcement member and to encapsulate the braided three-dimensional reinforcement member with a layer of the same or different fluid absorbing polymer.

21. A method of making a spinal nucleus implant according to claim 17 further comprising dehydrating the spinal nucleus implant to reduce the dimensions if the implant.

22. A method of making a spinal nucleus implant according to claim 21 wherein the spinal nucleus implant is subjected to equilateral radial compression to provide a bullet shaped dehydrated implant.

23. A method of treating a degenerating intervertebral disc comprising creating an incision in an annulus; removing at least a portion of a nucleus pulposus; and inserting, through the incision, a spinal nucleus implant according to claim 1.

24. A spinal nucleus implant comprising a three-dimensional reinforcement member adapted and configured to undergo anisotropic expansion, the implant configured to have a shape consistent with a cavity within an intervertebral disc space.

25. A spinal nucleus implant according to claim 24 wherein the three-dimensional reinforcement member is a braided fabric.

26. A spinal nucleus implant according to claim 24 further comprising a polymeric matrix.
